(19) United States
(12) Patent Application Publication
(10) Pub. No.: US 2011/0124019 A1
(43) Pub. Date: May 26, 2011

(54) METHOD OF USING CARBONIC ANHYDRASE TO DETECT HEMOLYSIS

(75) Inventors: Jeffrey A. Kline, Charlotte, NC (US); John Zagorski, Charlotte, NC (US)

(73) Assignee: The Charlotte-Mecklenburg Hospital Authority, Charlotte, NC (US)

(21) Appl. No.: 12/951,540

(22) Filed: Nov. 22, 2010

Related U.S. Application Data
(60) Provisional application No. 61/281,998, filed on Nov. 25, 2009.

(51) Int. Cl. G01N 33/566; C12Q 1/527

(52) U.S. Cl. 435/7.92; 435/4

(57) ABSTRACT

A method and a test for using carbonic anhydrase (CA), particularly CA-I or CA-II, as a biomarker of hemolysis. The method and test detect hemolysis by determining a percentage erythrocyte hemolysis in a specimen or sample of blood based upon quantification of carbonic anhydrase present in the extracellular portion of the blood. The method and test serve to optimize therapeutic efficacy for treatments of hemolysis. Plasma carbonic anhydrase is used to determine the percentage hemolysis in plasma. Furthermore, CA is quantified with specificity to the isozyme present in the plasma.

### CROSS-REFERENCE TO RELATED APPLICATIONS

This application claims the benefit of U.S. Provisional Patent Application No. 61/281,998, filed Nov. 25, 2009, herein incorporated by reference.

### FIELD OF THE INVENTION

The present invention relates to a method and a test to detect erythrocyte hemolysis using carbonic anhydrase, more particularly carbonic anhydrase I or II.

### BACKGROUND OF THE INVENTION

Carbonic anhydrase (CA) represents the second most abundant protein present inside the erythrocytes. With the exception of the hagfish and other lampreys, carbonic anhydrase activity or content is normally extremely low in vertebrates in the plasma. Carbonic anhydrase exists in at least twelve different isozymes, with variable distribution among tissues. The CA-I and CA-II isozymes predominate in erythrocytes, with a small amount of CA-III. Isozymes CA-IV to XII are found in many other tissues, including the gut, kidney and lung. Indeed, CA-IX is under study as a potential biomarker of renal and squamous cell carcinoma.

However, the characteristics and fate of free CA in plasma are poorly understood. Roush and Fiercke reported that free CA-I, CA-II, and CA-III in plasma is rapidly bound and inactivated by a transferrin-like protein, followed by clearance in the reticuloendothelial system. Studies of radiolabelled CA in rats demonstrates an approximate half-life of two hours with elimination in the kidney and liver for CA isozymes I and II.

It is known to those skilled in the art that the activity of carbonic anhydrase can be measured by colorimetric assays, typically employing a substrate that is recognized and cleaved by the active site of CA. One such example is the compound para nitro phenylacetate. Since carbonic anhydrase has intrinsic esterase activity, it will cleave the dye (p-nitrophenol) from the acetate liberating a color in aqueous solution that can be assayed by standard colorimetric techniques. However, such a method has multiple limitations. First, the plasma of mammals contains other esterases that will hydrolyze the substrate molecule and liberate the dye (example given, acetyl cholinesterase), this enzyme assay liberates very non-specific results. Other problems with the enzymatic assay include the fact that CA is inhibited by circulating proteins, as well as drugs, including sulfanilimides, ethanol and morphine.

Existing methods used to detect hemolysis have many shortcomings, most notably the inability to accurately quantify the percentage of erythrocytes hemolyzed based on a sample volume basis.

Existing methods indirectly point toward presence of hemolysis by measuring the disappearance plasma haptoglobin or appearance of bilirubin, a product of enzymatic oxidation of the heme moiety of hemoglobin by the enzyme hemeoxygenase. Erythrocyte destruction results in the release free hemoglobin, the most abundant protein in red blood cells, into the plasma. Free hemoglobin is rapidly and avidly bound by the circulating protein haptoglobin and to a lesser extent, hemopexin. The haptoglobin-hemoglobin is then bound to the scavenger receptor, CD-163, present on macrophages, monocytes, liver and spleen, and is removed from the circulation. As a result, the haptoglobin decreases in the presence of hemolysis. The limitations of haptoglobin are that a clinician can only know if the haptoglobin is lower than normal, and the clinician is almost never aware of the patient's baseline haptoglobin concentration. Moreover, haptoglobin synthesis is induced by many inflammatory conditions, including infection, cancer and drugs—the very stimuli that can actually produce hemolysis, such that a normal haptoglobin concentration can occur even with significant hemolysis. Thus, haptoglobin provides limited information about the degree of hemolysis.

It is well recognized that free plasma hemoglobin can be detected by spectrophotometric methods as well as immunoassay. However, because free hemoglobin is rapidly bound to haptoglobin and removed, it is not a reliable measurement of hemolysis produced by disease. Similarly, potassium is released from erythrocytes, but it is rapidly equilibrated within other cells of the body and therefore cannot be used to quantify the percentage hemolysis. Measurement of elevated plasma concentrations of unconjugated bilirubin and other products of the Heme oxygenase enzyme pathway also indicate evidence of hemolysis. However, the production of bilirubin from hemolysis varies depending upon the amount and activity of the heme oxygenase enzyme. Because heme oxygenase is highly inducible, and dependent upon certain genotypes, its activity cannot be predicted. The rate of elimination of unconjugated bilirubin is also difficult to predict because this depends upon hepatic function, blood flow and nutritional status. This, bilirubin does not suffice to quantify the degree of hemolysis.

Accordingly, there is a need for a test and for a method to detect hemolysis and to optimize therapeutic efficacy that overcomes the disadvantages discussed herein.

### SUMMARY OF THE INVENTION

The present invention is directed to a method and a test for using carbonic anhydrase, particularly CA-I or CA-II, as a biomarker of hemolysis. Thus, the present invention relates to a method and to a test for detecting hemolysis by determining a percentage erythrocyte hemolysis in a specimen or sample of blood based upon quantification of carbonic anhydrase (CA) present in the extracellular portion of the blood. The method and test serve to optimize therapeutic efficacy for treatments of hemolysis. Plasma carbonic anhydrase is used to determine the percentage hemolysis in plasma. Furthermore, for the purpose of this invention, CA is quantified with specificity to the isozyme present in the plasma.

It is within the scope of the present invention to provide a test or an assay that results in a color change that can be optically measured and the result converted from a nomogram or formula into a percentage hemolysis in a sample. This percentage can be used to optimize the efficacy of treatments for hemolysis. This percentage can be understood by the following hypothetical consideration. Consider a tube of 99 mL of plasma, completely devoid of any volume of erythrocytes. Thus, tube has 0% hemolysis. A second tube of 1 mL of packed red blood cells (PRBCs), which are for all practical purposes 100% erythrocytes by volume. If the second tube is subjected to high energy sonication, the erythrocytes will rupture, and the tube will contain 100% hemolysate. If 1 mL of this hemolysate is added to tube 1, then tube 1 would then contain 1% hemolysis by volume.

The significance of a test to quantify the percentage of hemolysis in a sample would have applicability to many diseases. These include genetic hemoglobinopathies, inherited abnormalities of the erythrocyte, including spherocytosis, paroxysmal nocturnal hemoglobinuria, thalassemias, disseminated intravascular coagulation secondary to infection, trauma, or cancer, erythrocyte destruction secondary to immune response to drugs, viral infections, and other stimuli, erythrocyte destruction secondary intravascular devices such as heart valves or from pulmonary hypertension.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, which are not necessarily to scale, wherein:

FIG. 1 is a graph of a standard curve for CA-1 illustrating CA-1 protein concentration (ng/well) versus optical density (OD) value at 450 nm.

FIG. 2 is a graph of a monoclonal Ab ELISA to detect CA-1 in human plasma containing a known amount of hemolysate illustrating percent (%) hemolysis versus optical density (OD) at 450 nm. FIG. 2 illustrates the nomogram principle wherein the degree of color change corresponds to the percentage of hemolyzed cells in a sample.

FIGS. 3A and 3B illustrate plots of two sets of data as set forth in Example 5.

### DETAILED DESCRIPTION OF THE INVENTION

The following detailed description of the embodiment(s) is merely exemplary in nature and is in no way intended to limit the invention, its application, or uses.

A blood sample is obtained from a subject with minimal iatrogenic hemolysis. Accordingly, it is advisable that the blood sample should be taken from as large bore an IV as possible, preferably 18 gauge or larger. Moreover, the blood sample is centrifuged to separate the erythrocyte from the plasma or serum component. Further, this serum or plasma component can be assayed for its potassium concentration, and if the potassium concentration is above 5.5 mEq/L, this could be strongly suggestive of erythrocyte destruction that occurred during the venipuncture and blood handling process. Specimens with serum or plasma potassium concentration above 5.5 mEq/L should be interpreted with caution or should be discarded.

Since the carbonic anhydrase enzyme exists in multiple isoenzymes that are produced by different tissues, it is important in accordance with the present invention to focus on carbonic anhydrase that is present in the erythrocytes.

One method of overcoming the limitations of known enzyme assays is to employ an antibody derived from a monoclonal cell line with a Fab portion that specifically recognizes the amino acid sequence unique to either carbonic anhydrase isoenzyme I or isoenzyme II, and to perform a standard enzyme-linked immunosorbent assay (ELISA).

In accordance with the present invention, the method of the present invention determines the percentage hemolysis in plasma by quantifying the amount of carbonic anhydrase I activity. The method generally comprises the following method steps. A blood sample is obtained from a subject, preferably a human subject having minimal iatrogenic hemolysis. The blood sample is centrifuged to separate erythrocyte from the plasma or serum component of the blood. An anti carbonic anhydrase I antibody or molecule similarly capable of specific binding to carbonic anhydrase such as a DNA aptamer, is immobilized on a durable surface including glass, plastic, polycarbonate, or other material to allow secondary optical interogation. Examples of this immobile surface include the bottom of a well in a microplate commonly used for ELISA detection of analytes or an immobilation platform to allow reflection absoprtiometry or fluorometric detection (e.g., the Luminex® detection system). Alternatively, the concentration could be determined by analysis of the spectra produced by bombarding a portion of the plasma with a beam of electrons and using mass spectrometry to analyze the ionized peptide fragments (see example table set forth herein demonstrating the results of plasma from rats with experimental pulmonary embolism). The plasma fraction, separated by centrifugation of a specimen of blood is added to a vessel that contains the surface with the immobilized antibody. A polyclonal antibody, or similar capture molecule, directed against carbonic anhydrase I containing a biotin label or other fluorescent, colorimetric or chemical property is added to the vessel. The optical density is measured at the appropriate wavelengths depending upon the absorption spectrum of the detection label. Alternatively, carbonic anhydrase could be determined by mass-based methods including altered resonance properties caused by antibody-capture of carbonic anhydrase on a piezoelectric material. Alternatively, carbonic anhydrase could be detected by reflection absorptiometry accomplished by measuring the change in an optical transmissive or reflective property conferred by the direct binding of carbonic anhydrase to a capturing molecule bound to an immobile surface.

The optical density is plotted on a nomogram or nomograph. Alternatively, the optical density is calculated using an equation to estimate the percent hemolysis present in the plasma fraction or component. The calculation may be automated, for example, using a computer or other device to run a computerized analysis.

The method further comprises converting the amount of carbonic anhydrase activity into a percentage hemolysis in the plasma fraction using the nomogram or the equation.

The percentage of erythrocytes hemolyzed is determined by measuring the percentage hematocrit in a noncentrifuged blood specimen and employing an equation or formula containing the variables hematocrit and percentage hemolysis.

The percentage hemolysis is used to diagnose and monitor diseases and conditions that produce intravascular hemolysis as well as to optimize the therapeutic efficacy of treatment for hemolysis. Carbonic anhydrase (CA isoforms I and II), exists in high concentrations in erythrocytes, but not in plasma. In accordance with the present invention, it was determined that the method of the present invention uses the CA present in plasma as a quantitative biological indicator of intravascular hemolysis.

The present invention also relates to a test or kit for the detection of hemolysis up to about at least about 5 to 6%. This is a very important range clinically because, for example, at concentrations such as 8 to 10% the plasma begins to turn pink which is a visible indicator of free hemoglobin. The test or kit of the present invention is useful to identify patients with chronic diseases who are susceptible to hemolysis, and possibly to discover it earlier. For example, the test or kit could be used for various medical applications including, but not limited to, monitor patients on drugs that predispose to hemolysis as well as patients with hemoglobinopathies, patients at risk for disseminated intravascular coagulation, monitor malaria, assess severity of pulmonary hypertension, and for the initial work-up for anemia.

The present invention also relates to a method of diagnosing or treating a patient. The method comprises obtaining a percentage of hemolysis from a nomogram comprising a first set of data corresponding to optical density of a plasma component of a blood sample obtained from a subject and a second set of data corresponding to carbonic anhydrase I activity. The first set of data and the second set of data are plotted to form a graphical measure or an equation used to determine the percentage of hemolysis in the sample. The obtained percentage of hemolysis is used to diagnose or to treat the patient. For example, in the case of treatment by dosage of medication, a value of 0% hemolysis indicates a zero dosage amount, a value above 5% hemolysis indicates full dosage, and a value between 0 and 5% hemolysis indicate partial dosage, determined in proportion to the percentage of hemolysis obtained.

### Example 1

Standard solutions for analysis were prepared as follows. The percentage hemolysis was determined experimentally by carefully drawing a blood sample from a human that produced no hemolysis during phlebotomy portion. The sample was centrifuged at 3500×g for 10 min at 0° C. The plasma fraction with no hemolysis was removed and the hemolysate was produced from the packed red blood cells (PRBC) fraction by combining 0.5 mL of PRBCs with 0.75 mL of distilled water followed by sonication for 4 minutes. The resultant mixture was assumed to contain 40% hemolysate, and was then centrifuged and combined with neat plasma to produce known 0%, 0.1%, 2.5%, 5%, and 10% hemolysate. A standard curve was based upon purchased human CA-1 protein (Sigma C-4396) dissolved in distilled water.

In initial experiments, a polyclonal anti-CA antibody was tested and found extremely high background color change in plasma containing 0% hemolysate, indicating non-specific binding, and compelling the need for a monoclonal antibody directed against CA-1.

### Example 2

An experiment was conducted in accordance with the present invention using a purchased monoclonal antibody against carbonic anhydrase isoenzyme I (CA-I) in humans. The antibody was purchased from a commercial source (Abcam). This monoclonal, anti human CA-I antibody represented the capturing antibody. A 1:10,000 dilution of the monoclonal anti-CA I antibody was incubated in a phosphate-containing buffer for sufficient time to allow binding to the bottom of the wells of a standard 96-well micro plate. This antibody served as the CA-I capture antibody. In the next step, human plasma was introduced that had successive concentrations of hemolysis. The detection antibody consisted of a biotinylated polyclonal anti-CA-I polyclonal antibody (purchased from Abcam, in a 1:5,000 dilution) and the biotin label was detected by a chromogenic tag.

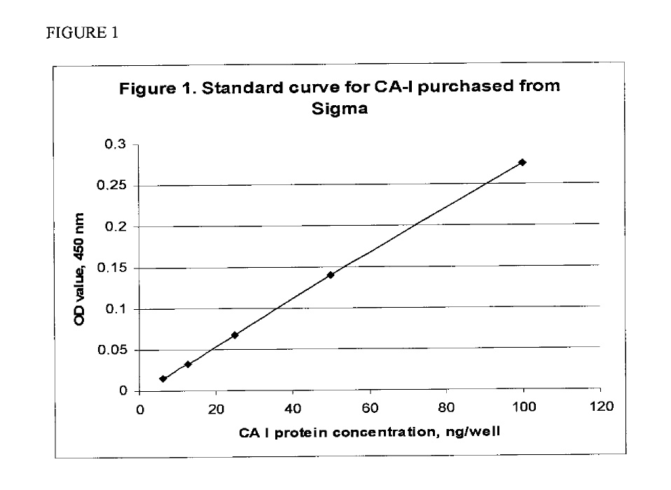

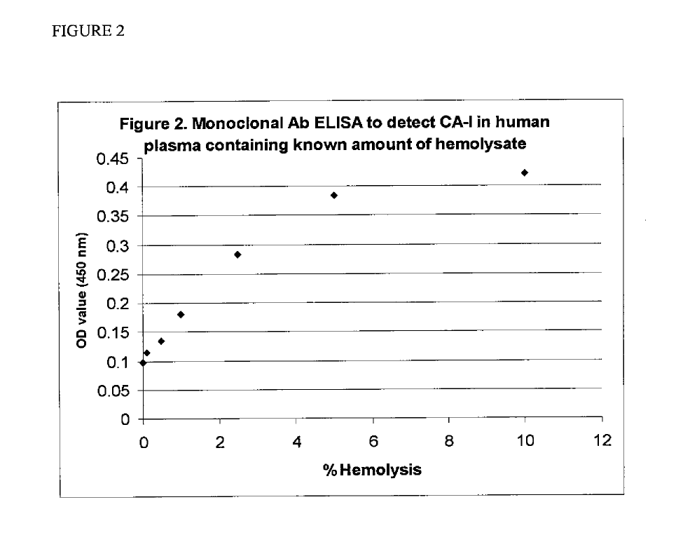

FIG. 1 is a graph illustrating the result of the standard curve comparing the optical density value produced by the chromogenic tag at 450 nanometers. FIG. 2 illustrates the curve that was obtained by measuring the optical density of plasma containing no hemolysis, followed by the concentrations previously mentioned. FIG. 2 illustrates the nomogram principle wherein the degree of color change corresponds to the percentage of hemolyzed cells in a sample. It can be observed that the response was linear up to approximately a concentration of about 5% total hemolysis. Between about 5 to 10%, a nearly horizontal asymptote was observed.

It was determined that the equation that described the best fit straight line in FIG. 2 from 0 to 5% hemolysis was:

OD=0.057(% hemolysis)+0.1 EQUATION 1

It is within the scope of the present invention that this equation may vary somewhat depending upon experimental conditions. However, in accordance with the present invention, a nomogram is obtained to convert the OD reading into percent hemolysis using, for example, a visual aid, computer or other device. For example, a sample of plasma that is diluted in buffer by a 1:5 ratio yields an OD of 0.2. Solving equation 1 would produce a % hemolysis=((0.2−0.1)/0.057)*5=8.7%.

It is also evident that the method of the present invention could be used to determine the percentage hemolysis in the plasma fraction. The percentage of the erythrocyte volume that was hemolyzed could be estimated by determining the percentage hemolysis using the volume of erythrocytes derived from the hematocrit (Hct). The formula for estimating the percentage of erythrocytes hemolyzed is % EH=[(% hemolysis in plasma)*(1−Hct)/Hct]*100%. Thus, for a specimen with a 30% hematocrit and a 4% hemolysis measured in the plasma fraction, % EH=100*(0.04)*(0.7)/0.3=9%.

### Example 3

TABLE 3-1

| Blood samples | OD1 | OD2 |
|---|---|---|
| plasma, 0% | 0.077 | 0.071 |
| plasma, 0.1% | 0.062 | 0.078 |
| hemolysis | | |
| plasma, 1% | 0.103 | 0.095 |
| hemolysis | | |
| plasma, 2.5% | 0.162 | 0.16 |
| hemolysis | | |
| plasma, 5% | 0.273 | 0.279 |
| hemolysis | | |
| plasma, 10% | 0.398 | 0.362 |
| hemolysis | | |
| plasma, 20% | 0.299 | 0.316 |
| hemolysis | | |

OD1 and OD2 are replicated values from the same sample.

TABLE 3-2

| Protein Standard, | | |
|---|---|---|
| ug/well | OD1 | OD2 |
| 0.1 | 0.391 | 0.352 |
| 0.2 | 0.586 | 0.546 |
| 0.4 | 0.746 | 0.694 |
| 0.6 | 0.703 | 0.721 |
| 1 | 0.881 | 0.778 |

OD1 and OD2 are replicated values from the same sample.

Capturing Antybody (1st)—anti-CAI monoclonal Ab (Abcam)

Detection Antybody—biotinilated anti-CAI polyclonal Ab (Abcam)

Blood Samples Preparation:

1. Whole blood was collected from a healthy human into 4×3 ml vacuettes (contain 3.2% Sodium Citrate)

2. Plasma and RBC were separated buy centrifugation for 10 min at 2500 g.

3. 0.5 ml RBC were combined with 0.75 ml of distilled H₂O and sonicated for 4 min in the water bath sonicator.

This RBC preparation equaled 40% hemolysis.

4. Plasma and hemolysed RBCs were combined to produce the following concentrations of hemolysis in plasma: 0.1%, 1%, 2.5%, 5%, 10%, 20%.

### Example 4

TABLE 4-1

| Blood samples | OD | OD | OD | OD average |
|---|---|---|---|---|
| plasma, 0% | 0.095 | 0.122 | 0.075 | 0.097333333 |
| plasma, 0.1% | 0.097 | 0.133 | 0.113 | 0.114333333 |
| hemolysis | | | | |
| plasma, 0.5% | 0.114 | 0.159 | 0.129 | 0.134 |
| hemolysis | | | | |
| plasma, 1% | 0.176 | 0.192 | 0.173 | 0.180333333 |
| hemolysis | | | | |
| plasma, 2.5% | 0.24 | 0.341 | 0.267 | 0.282666667 |
| hemolysis | | | | |
| plasma, 5% | 0.422 | 0.395 | 0.335 | 0.384 |
| hemolysis | | | | |
| plasma, 10% | 0.334 | 0.464 | 0.465 | 0.421 |
| hemolysis | | | | |

TABLE 4-2

| Protein | | | | |
|---|---|---|---|---|
| concentration, | | | | |
| ng/well | OD average | OD | OD | OD |
| 6.25 | 0.014666667 | 0.017 | 0.015 | 0.012 |
| 12.5 | 0.032 | 0.044 | 0.025 | 0.027 |
| 25 | 0.067666667 | 0.087 | 0.062 | 0.054 |
| 50 | 0.14 | 0.159 | 0.157 | 0.104 |
| 100 | 0.275 | 0.241 | 0.354 | 0.23 |
| 200 | 0.438666667 | 0.463 | 0.493 | 0.36 |

Capturing Antibody (1st)—anti-CA I monoclonal Ab (Abcam). 1:10000 dilution

Detection Antibody—biotinilated anti-CA I polyclonal Ab (Abcam). 1:5000 dilution

Blood Samples Preparation:

1. Whole blood was collected from a healthy human into 5×3 ml vacuettes (contain 3.2% Sodium Citrate).

2. Plasma and RBC were separated by centrifugation for 10 minutes at 2500 g.

3. 0.5 ml RBC were combined with 0.75 ml of distilled H₂O and sonicated for 4 minutes in the water bath sonicator.

This RBC preparation equals 40% hemolysis.

4. Plasma and hemolysed RBCs were combined to produce the following concentrations of hemolysis in plasma: 0.1%, 0.5%, 1%, 2.5%, 5%, 10%.

5. CA I protein standard was made by preparing a stock of CA I (Sigma C-4396) in dH₂0 at 200 ug/ml. Further dilutions were made accordingly.

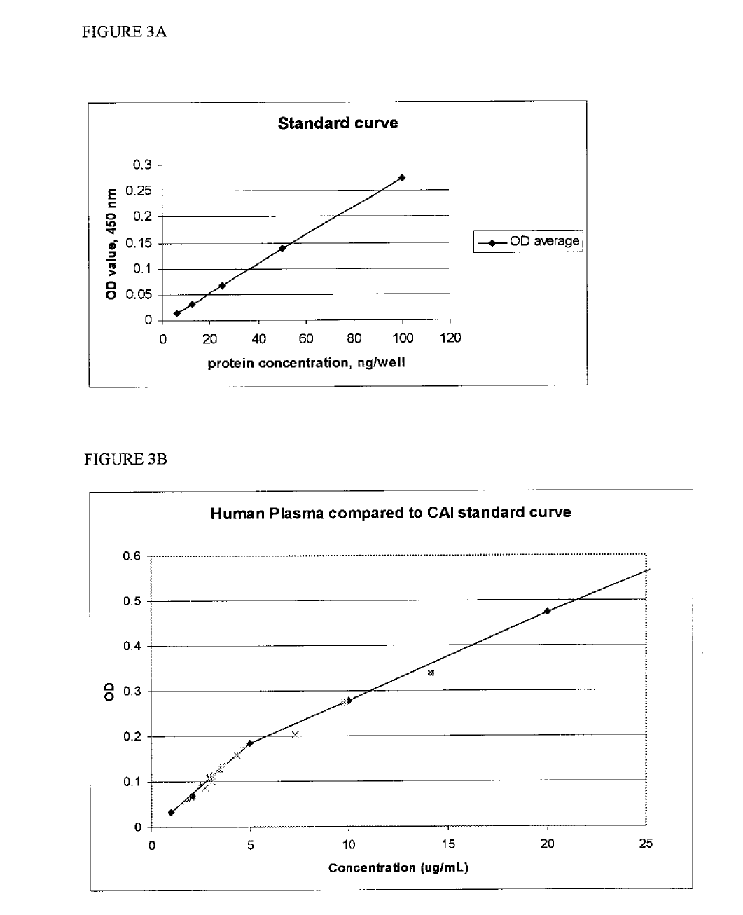

FIGS. 3A and 3B illustrate the resultant standard curve.

### Example 5

The following Carbonic Anhydrase I (CA-I) ELISA with monoclonal AB was conducted. The following materials were used:

96-well ELISA plates (Nunc 439454)

Monoclonal capture Ab (mouse anti-CA I, Abcam, Inc. #70418, −20° C. in yellow box labeled “MO_CAI”)

Capture Ab dilution buffer, 0.1M NaHCO₃

Refrigerator set to 4° C.

Plate shaker in cold room set to 79 RPM

Incubator set to 37° C.

Wash buffer, 1×PBS+0.05% Tween-20

Blocking buffer, 1×PBS+0.05% Tween-20+1% BSA

1×PBS

Samples

Standard, CA I Enzyme (Sigma C-4396; stock at 1 mg/mL in water, −20° C.)

Biotinylated polyclonal detector Ab (goat anti-CA I, Abcam, Inc. #34567, −20° C. in yellow box labeled “MO_CAI”)

Detector Ab dilution buffer, 1×PBS+1% BSA

streptavidin-HRP (R&D Systems cat #DY998, 4° C.)

Substrate, (R&D Systems cat # DY999, 4° C.)

Stop solution (1 sulfuric acid: 4 H₂O, i.e. 3 ml stock sulfuric acid+9 ml tap H₂O. Invert to mix and cool down at −20° C. prior to use)

The following procedure was followed:

Capture Ab was diluted 1:10,000 in Capture Ab dilution buffer. 1004, was added to all wells that were used. Incubated overnight at 4° C. on a plate shaker.

The plate was washed four times with Wash buffer, using a multi-channel pipette.

200 μL/well of Blocking buffer was added. Incubated at 4° C. for 2 hours.

Washed as in step 2.

Samples and protein standard, 100 μL/well were added. Blank was PBS. Incubated at 37° C. for 90 min. 200 μL CA I enzyme was added to the first well of the standard row. 100 μL PBS was added to the remaining wells that contained standard. Pipette 100 μL from first well and added to next well in the same row. Pipette used to mix and took 100 μL from that well and added to the next. Repeated down the row to make serial dilutions of the CA I standard; prepared dilutions fresh each time.

Washed as in step 2.

Detector Ab was diluted 1:5,000 (1:2,500 when stored 1:1 in glycerol) in Detector Ab dilution buffer. 100 μL/well added. Incubated for 1 hour at 4° C. on a plate shaker.

Washed as in step 2.

Diluted streptavidin-HRP 1:200 in Blocking buffer. Added 100 μL/well. Incubated at 4° C. for 30 minutes.

Washed as in step 2.

Washed 3 times with 1×PBS.

Removed substrate from 4° C. and warmed to room temperature. Made a 1:1 dilution of reagent A and reagent B. Added 100 μL/well. Incubated 30 min at room temperature. Stopped reaction with 50 μL/well of stop solution. Reaction turned yellow. Read at 450 nm.

Note that the plate was protected from light by wrapping in foil while incubating.

The following results were obtained following the above procedure.

TABLE 5-1

| | Standard concentration (ug/mL) | | | | | | | | | | | |
|---|---|---|---|---|---|---|---|---|---|---|---|---|
| | 100 | 90 | 80 | 70 | 60 | 50 | 40 | 30 | 20 | 10 | 5 | 1 |
| Standard curve | 0.999 | 1.072 | 1.059 | 1.196 | 0.995 | 0.869 | 0.661 | 0.652 | 0.474 | 0.279 | 0.185 | 0.032 |

TABLE 5-2

| | | Concentration |
|---|---|---|
| Biosite Plasma Sample # | OD | (ug/mL) |
| 8 | 0.336 | 14.18 |
| 9 | 0.067 | 2.09 |
| 10 | 0.203 | 7.28 |
| 11 | 0.108 | 2.92 |
| 12 | 0.067 | 2.09 |
| 13 | 0.093 | 2.51 |
| 14 | 0.123 | 3.32 |
| 15 | 0.134 | 3.62 |
| 16 | 0.133 | 3.59 |
| 17 | 0.137 | 3.7 |
| 18 | 0.165 | 4.46 |
| 19 | 0.099 | 3.09 |
| 20 | 0.111 | 3 |
| 21 | 0.132 | 3.58 |
| 22 | 0.051 | 1.59 |
| 23 | 0.058 | 1.81 |
| 24 | 0.173 | 4.68 |
| 25 | 0.114 | 3.08 |
| 26 | 0.273 | 9.78 |
| 27 | 0.127 | 3.43 |
| 28 | 0.087 | 2.72 |
| 29 | 0.160 | 4.32 |

FIGS. 3A and 3B demonstrate plots of two sets of data, one being a standard curve using commercially obtained CA-I (represented by the closed diamonds, in both plots 3A and 3B). FIG. 3A shows the linearity across a wide concentration of CA-I. FIG. 3B shows the standard curve at a clinically significant CA-I concentration range, (plotted with closed diamonds) and the line representing the best fit straight line. All other symbols representing the measured CA-I values from plasma samples from 22 humans. These data correspond to Tables 5-1 and 5-2.

The mean was 4.1291 and the standard deviation (SD) was 2.8882. It was concluded from this data that a value greater than (4.1+(2.8×2)) or greater than about 10 ug/mL is abnormally high at the 95 percentile. It was also concluded that an OD of about 0.25 to 0.30 indicated a CA I concentration of about 10 ug/mL or higher. Thus, OD values of about 0.2 to 0.3 corresponded to the lower limit range that signifies a clinically significant level of intravascular hemolysis. Optical density values above 0.3 indicated worsened burden of hemolysis and indicated a clinically significant level of hemolysis. Optical density values below 0.2 indicated less hemolysis and indicated a clinically insignificant level of hemolysis.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

What is claimed is:

1. A method for detecting hemolysis in a subject, the method comprising:
correlating optical density of a plasma component of a blood sample with carbonic anhydrase concentration to determine a percentage hemolysis present in the plasma component of the blood sample.

2. The method according to claim 1, wherein correlating comprises plotting the optical density on a nomogram.

3. The method according to claim 1, wherein correlating comprises calculating the optical density using an equation to estimate the percentage hemolysis present in the plasma component.

4. The method according to claim 3, wherein the calculation is automated using a computer or other device to run a computerized analysis.

5. The method according to claim 1, wherein the carbonic anhydrase concentration is of carbonic anhydrase I or carbonic anhydrase II.

6. The method according to claim 1, wherein concentration is determined by analysis of spectra produced by bombarding a portion of the plasma with a beam of electrons.

7. A method for detecting hemolysis in a subject, the method comprising:
assaying a plasma component separated from a blood sample obtained from a subject, and
correlating optical density of the plasma component of the blood sample with carbonic anhydrase I concentration to determine a percentage hemolysis present in the plasma component of the blood sample.

8. The method according to claim 7, wherein the assay results in a color change that can be optically measured.

9. The method according to claim 7, wherein the plasma component is assayed by performing an enzyme-linked immunosorbent assay (ELISA) involving at least one antibody derived from a monoclonal cell line with a Fab portion that specifically recognizes an amino acid sequence unique to carbonic anhydrase I.

10. The method according to claim 9, wherein the antibody comprises a biotin label.

11. The method according to claim 9, wherein a fluorescent, colorimeter or chemical property is present in the assay.

12. The method according to claim 7, wherein the optical density of the plasma component of the blood sample is determined at a specified wavelength.

13. The method according to claim 7, wherein the optical density in a range of about 0.2 to 0.3 represents a range where the degree of hemolysis becomes clinically significant for diagnosis and treatment of the subject.

14. The method according to claim 7, wherein an optical density less than 0.2 indicates a clinically insignificant level of hemolysis.

15. The method according to claim 7, wherein an optical density greater than 0.3 indicates a clinically significant level of hemolysis.

16. A method for optimizing therapeutic efficacy associated with treatment of hemolysis in a subject, the method comprising:
determining a percentage of hemolysis in a plasma component separated from a blood sample obtained from a subject by correlating optical density of the plasma component of the blood sample with carbonic anhydrase I activity,
wherein the determined percentage of hemolysis is used to optimize the therapeutic efficacy associated with treatment of hemolysis in a subject.

17. A test for the detection of hemolysis in a subject, the test comprising:
a nomogram comprising a first set of data corresponding to optical density of a plasma component of a blood sample obtained from a subject and a second set of data corresponding to carbonic anhydrase I activity, wherein the first set of data and the second set of data are plotted to form a graphical measure or an equation used to determine the percentage of hemolysis in the sample.

18. The test according to claim 17, wherein the nomogram detects hemolysis up to about at least 5% to 6%.

19. A method for optimizing therapeutic efficacy associated with treatment of hemolysis in a subject, the method comprising:
obtaining a percentage of hemolysis from a nomogram comprising a first set of data corresponding to optical density of a plasma component of a blood sample obtained from a subject and a second set of data corresponding to carbonic anhydrase I activity, wherein the first set of data and the second set of data are plotted to form a graphical measure or an equation used to determine the percentage of hemolysis in the sample,
quantifying the percentage hemolysis based upon measurement of hematocrit, and
using a formula to estimate a percentage of erythrocytes hemolyzed in-vivo.

20. A method of diagnosing or treating a patient, the method comprising:
obtaining a percentage of hemolysis from a nomogram comprising a first set of data corresponding to optical density of a plasma component of a blood sample obtained from a subject and a second set of data corresponding to carbonic anhydrase I activity, wherein the first set of data and the second set of data are plotted to form a graphical measure or an equation used to determine the percentage of hemolysis in the sample, and
using the obtained percentage of hemolysis to diagnose or treat the patient.

21. The method according to claim 20, wherein for treatment by dosage of medication, a value of 0% hemolysis indicates a zero dosage amount, a value of above 5% hemolysis indicates full dosage, and a value between 0 and 5% hemolysis indicates partial dosage, determined in proportion to the percentage of hemolysis obtained.
